(19) United States
(12) Patent Application Publication
(10) Pub. No.: US 2026/0115386 A1
(43) Pub. Date: Apr. 30, 2026

(54) SYRINGES, SYSTEMS, AND METHODS OF USE

(71) Applicant: RENSSELAER POLYTECHNIC INSTITUTE, Troy, NY (US)

(72) Inventors: Richard KIM, Gansevoort, NY (US); Kelly GRAY, Troy, NY (US); Amanda RAMPADO, Troy, NY (US); Dianna STUZHUK, Troy, NY (US); Amanda SU, Troy, NY (US); Sara ZAFIAN, Troy, NY (US)

(73) Assignee: RENSSELAER POLYTECHNIC INSTITUTE, Troy, NY (US)

(21) Appl. No.: 19/369,871

(22) Filed: Oct. 27, 2025

Related U.S. Application Data
(63) Continuation of application No. PCT/US2024/026440, filed on Apr. 26, 2024.
(60) Provisional application No. 63/498,518, filed on Apr. 26, 2023.

(51) Int. Cl. A61M 5/31; A61J 1/20; A61M 1/36; A61M 5/315

(52) U.S. Cl. CPC A61M 5/3134; A61J 1/2048; A61M 1/3693; A61M 5/31511; A61M 2005/3104; A61M 2202/0415; A61M 2202/0427

(57) ABSTRACT

Syringes and syringe systems for use in centrifuge machines including coupling arms for engaging the centrifuge machines. The syringe including a housing with a first coupling arm extending from a first side of the housing and a second coupling arm extending from a second side of the housing. The first side is opposite the second side. The syringe also includes at least one plunger slidingly received within the housing. Methods of using the syringes and syringe system for extracting and injecting platelet-rich plasma are also disclosed.

### CROSS REFERENCE TO RELATED APPLICATIONS

This application is a continuation of International Application No. PCT/US2024/026440 filed Apr. 26, 2024 and entitled “Syringes, Systems, and Methods of Use,” which claims priority benefit under 35 U.S. C. § 120 of U.S. Provisional Application No. 63/498,518 filed Apr. 26, 2023, which are incorporated herein by reference in their entireties.

### TECHNICAL FIELD

The present disclosure relates generally to syringes for extraction of plasma and centrifuge of same. More specifically, but not exclusively, the present disclosure relates to syringes, systems, and methods for using same to extract plasma and centrifuge the platelet-rich plasma.

### BACKGROUND OF THE INVENTION

Currently available technology requires, at least, using a syringe to remove the blood from a patient and then placing the blood into a vial for insertion into a centrifuge to extract the platelet-rich plasma. In one embodiment, extracting platelet-rich plasma requires a twelve step procedure. The procedure requires taking blood from a patient with a needle and extracting the blood into vials. The vials can then be centrifuged to separate out the platelet-rich plasma. The plasma may be removed from the centrifuged vials. The centrifuge process is then repeated. Once the platelet-rich plasma is extracted it may be injected into a patient.

Thus, new and improved syringes, systems and methods for using the syringes are needed to improve the platelet-rich plasma extraction procedure.

### SUMMARY OF THE INVENTION

The present disclosure is directed toward syringes, systems, and methods for using the same to extract platelet-rich plasma.

In one aspect of the present disclosure provided herein, is a syringe. The syringe including a housing with a first coupling arm extending from a first side of the housing and a second coupling arm extending from a second side of the housing. The first side is opposite the second side. The syringe also includes at least one plunger slidingly received within the housing.

In another aspect of the present disclosure provided herein, is a syringe system. The syringe system including at least one first syringe. The at least one first syringe including a housing and at least one plunger slidingly received within the housing. The housing includes a first coupling arm extending from a first side of the housing and a second coupling arm extending from a second side of the housing. The first side is opposite the second side. The syringe system including at least one second syringe and at least one syringe cap for removable coupling to at least one of the at least one first syringe and the at least one second syringe.

In yet another aspect of the present disclosure provided herein, is a method of using a syringe system. The method includes obtaining a syringe system. The method also includes removing blood from a patient with a first syringe of the syringe system, wherein the first syringe includes two coupling arms positioned on an exterior surface of the first syringe opposite each other. The method further includes placing the syringe into a centrifuge machine and coupling the first syringe with a second syringe and extracting platelet-rich plasma from the first syringe.

These and other objects, features and advantages of this disclosure will become apparent from the following detailed description of the various aspects of the disclosure taken in conjunction with the accompanying drawings.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the disclosure.

FIG. 1 is a perspective view of one embodiment of a syringe, in accordance with an aspect of the present disclosure;

FIG. 2 is a side view of the syringe of FIG. 1, in accordance with an aspect of the present disclosure;

FIG. 3 is a perspective view of the syringe of FIG. 1 with a transparent housing, in accordance with an aspect of the present disclosure;

FIG. 4 is a side view of the syringe of FIG. 1 with a transparent housing, in accordance with an aspect of the present disclosure;

FIG. 5 is an exploded, first perspective view of the syringe of FIG. 1, in accordance with an aspect of the present disclosure;

FIG. 6 is an exploded, second perspective view of the syringe of FIG. 1, in accordance with an aspect of the present disclosure;

FIG. 7 is an exploded, top view of the syringe of FIG. 1, in accordance with an aspect of the present disclosure;

FIG. 8 is an exploded, bottom view of the syringe of FIG. 1, in accordance with an aspect of the present disclosure;

FIG. 9 is an exploded, first side view of the syringe of FIG. 1, in accordance with an aspect of the present disclosure;

FIG. 10 is an exploded, second side view of the syringe of FIG. 1, in accordance with an aspect of the present disclosure;

FIG. 11 is an exploded, front view of the syringe of FIG. 1, in accordance with an aspect of the present disclosure;

FIG. 12 is an exploded, back view of the syringe of FIG. 1, in accordance with an aspect of the present disclosure;

FIG. 13 is a cross-sectional perspective view of the syringe of FIG. 1 taken along line 13-13 in FIG. 1, in accordance with an aspect of the present disclosure;

FIG. 14 is a cross-sectional side view of the syringe of FIG. 13, in accordance with an aspect of the present disclosure;

FIG. 15 is a top transparent view of another housing of the syringe of FIG. 1, in accordance with an aspect of the present disclosure;

FIG. 16 is a side transparent view of the housing of FIG. 15, in accordance with an aspect of the present disclosure;

FIG. 17 is a cross-sectional side view of another syringe embodiment, in accordance with an aspect of the present disclosure;

FIG. 18 is a cross-sectional side view of yet another syringe embodiment, in accordance with an aspect of the present disclosure;

FIG. 19 is a perspective view of the syringe of FIG. 1 with the outer plunger removed, in accordance with an aspect of the present disclosure;

FIG. 20 is a side view of the syringe of FIG. 19, in accordance with an aspect of the present disclosure;

FIG. 21 is a perspective view of the syringe of FIG. 19 with a transparent housing, in accordance with an aspect of the present disclosure;

FIG. 22 is a side view of the syringe of FIG. 19 with a transparent housing, in accordance with an aspect of the present disclosure;

FIG. 23 is a cross-sectional perspective view of the syringe of FIG. 19 taken along line 23-23 in FIG. 19, in accordance with an aspect of the present disclosure; and

FIG. 24 is a cross-sectional side view of the syringe of FIG. 19, in accordance with an aspect of the present disclosure.

### DETAILED DESCRIPTION FOR CARRYING OUT THE INVENTION

Generally stated, disclosed herein are syringes for extraction of blood and centrifuging the same. More specifically, but not exclusively, the present disclosure relates to syringes, systems, and methods for using the same to extract blood and centrifuge the platelet-rich plasma from the blood.

In this detailed description and the following claims, the words proximal, distal, anterior, posterior, medial, lateral, superior and inferior are defined by their standard usage for indicating a particular part of a device according to the relative disposition of the device with respect to a body or directional terms of reference. For example, “proximal” means the portion of a device nearest the point of injection, while “distal” indicates the portion of the device farthest from the point of injection. As for directional terms, “anterior” is a direction towards the front side of the device, “posterior” means a direction towards the back side of the device, “medial” means towards the midline of the device, “lateral” is a direction towards the sides or away from the midline of the device, “superior” means a direction above and “inferior” means a direction below another object or structure.

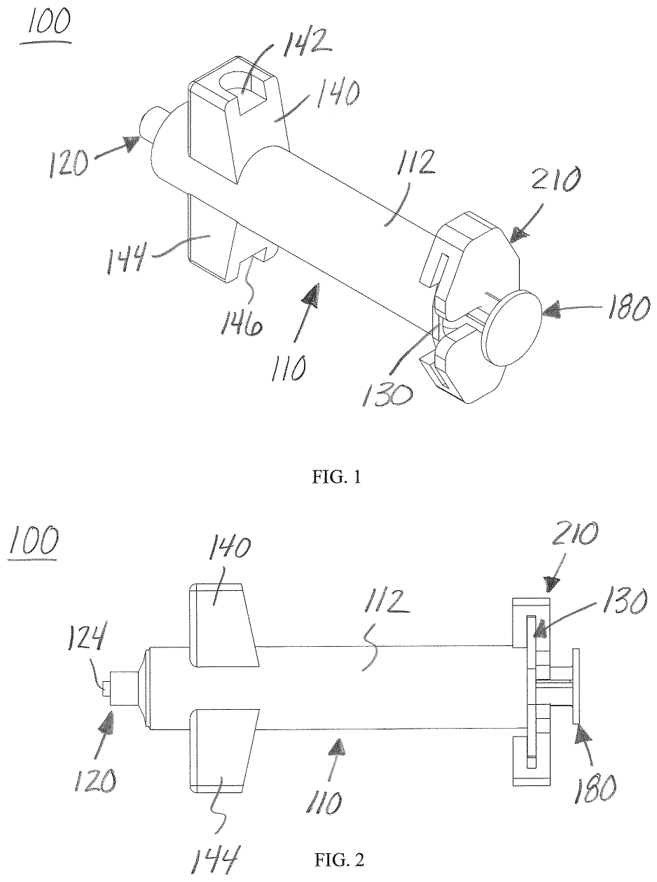

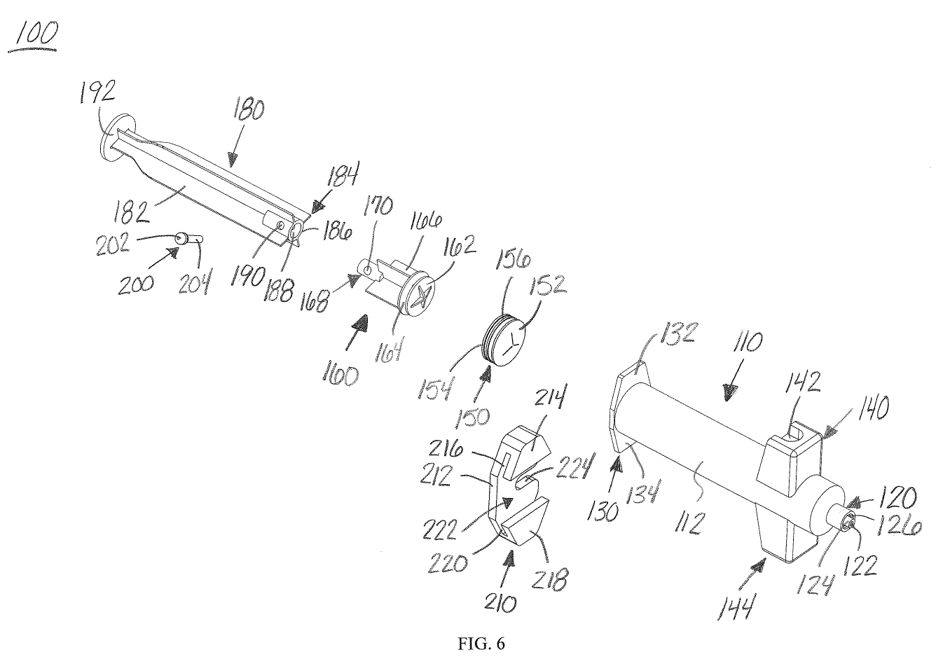

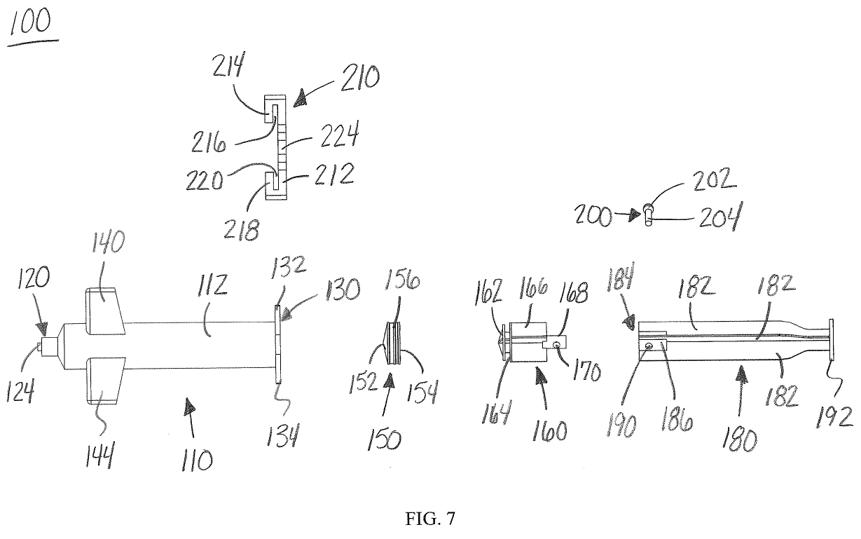

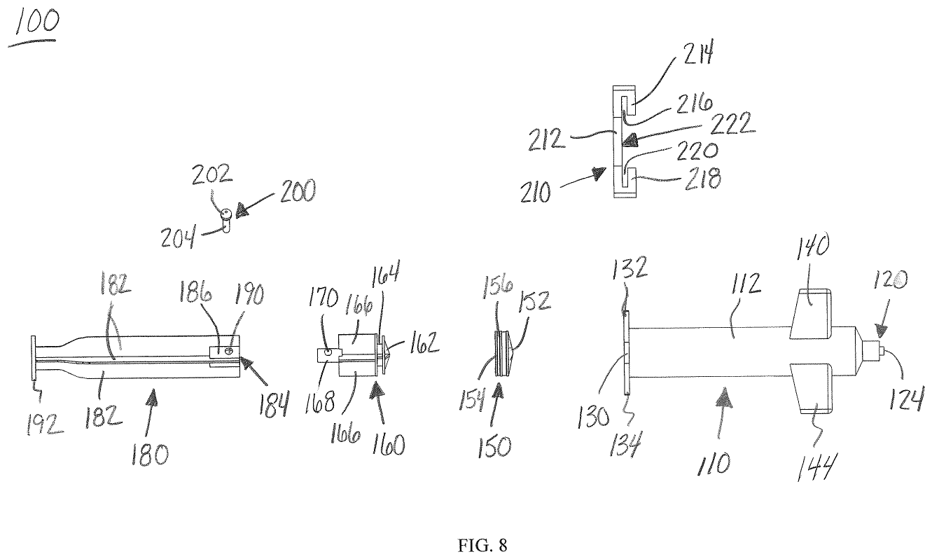

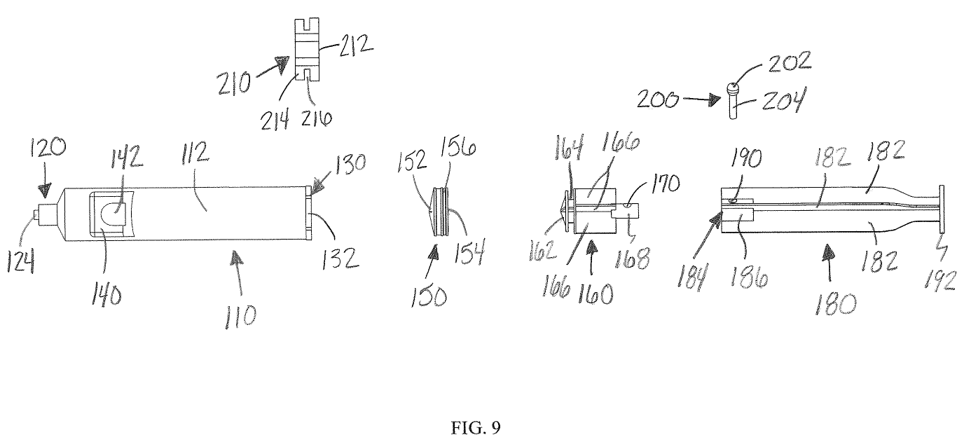

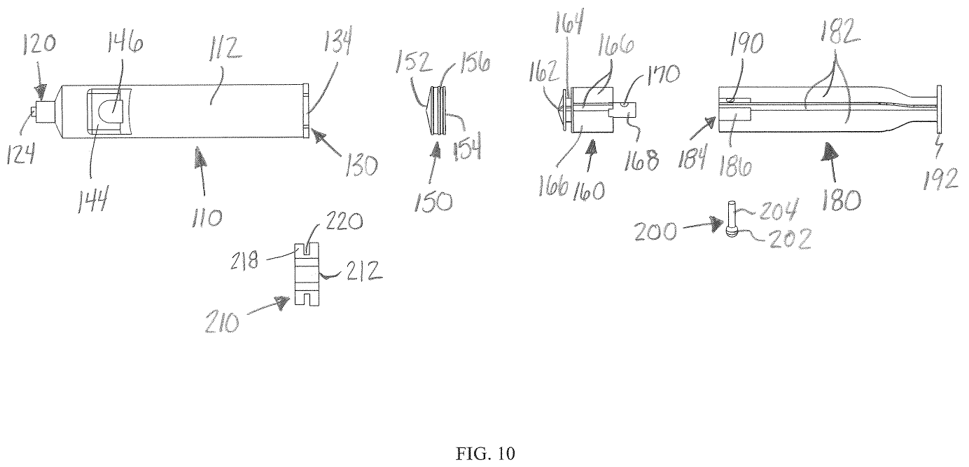

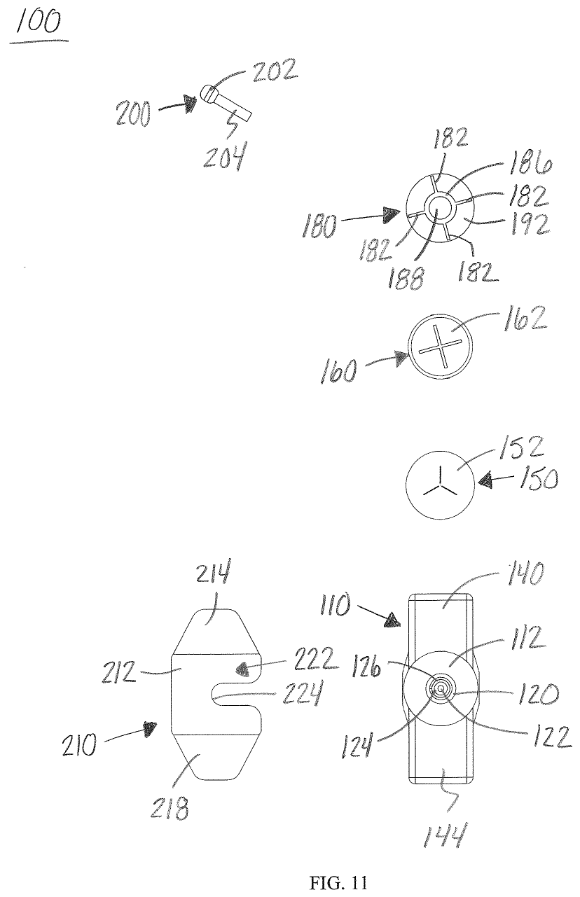

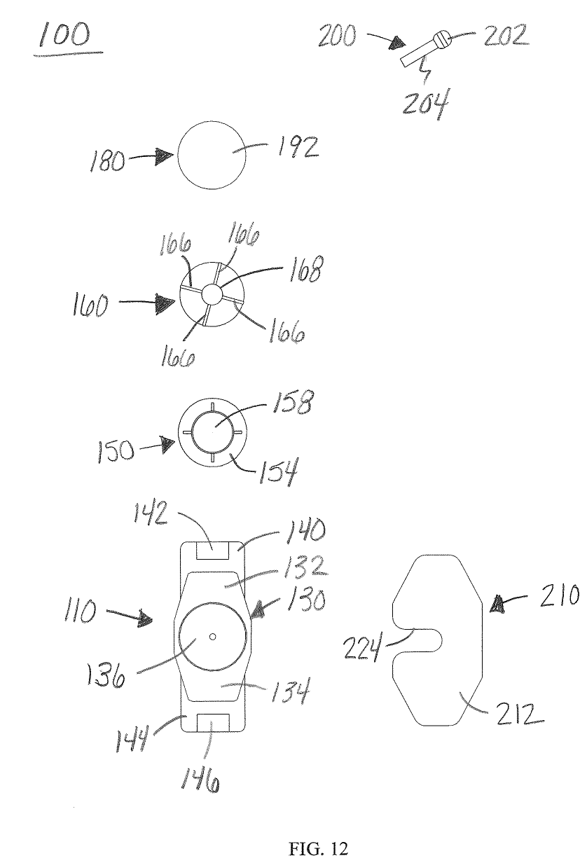

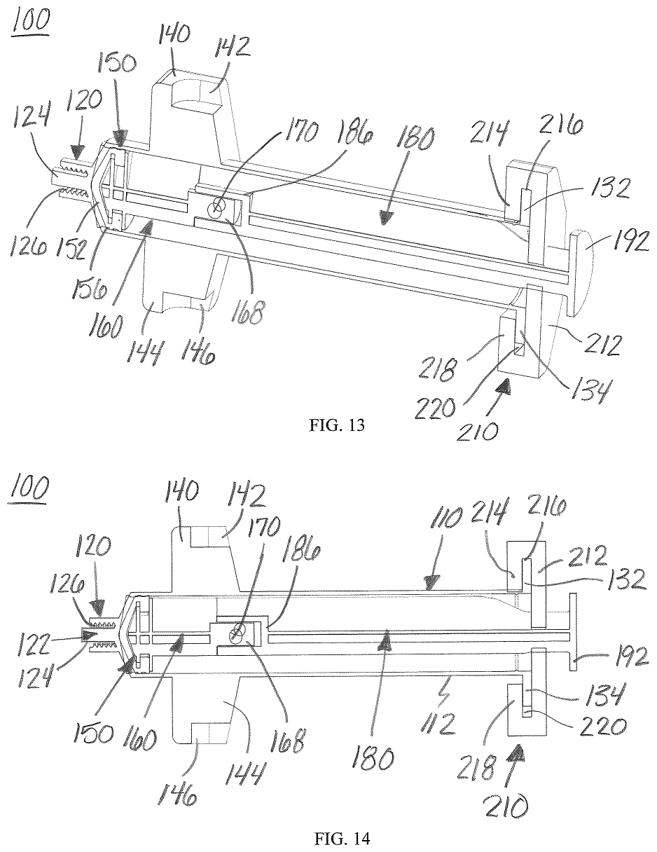

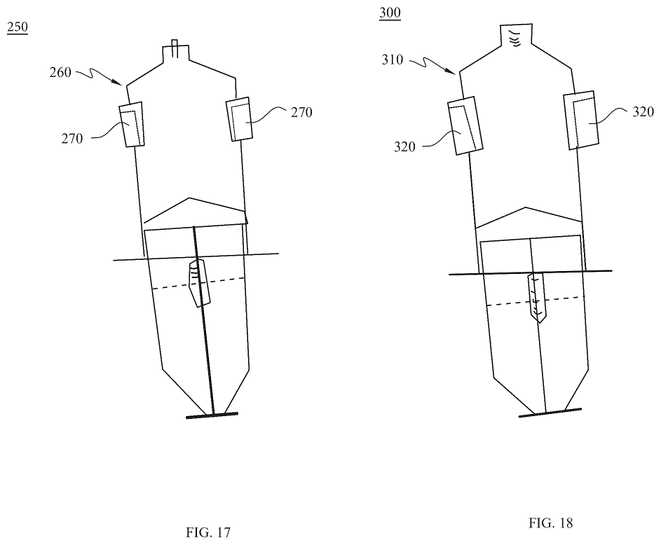

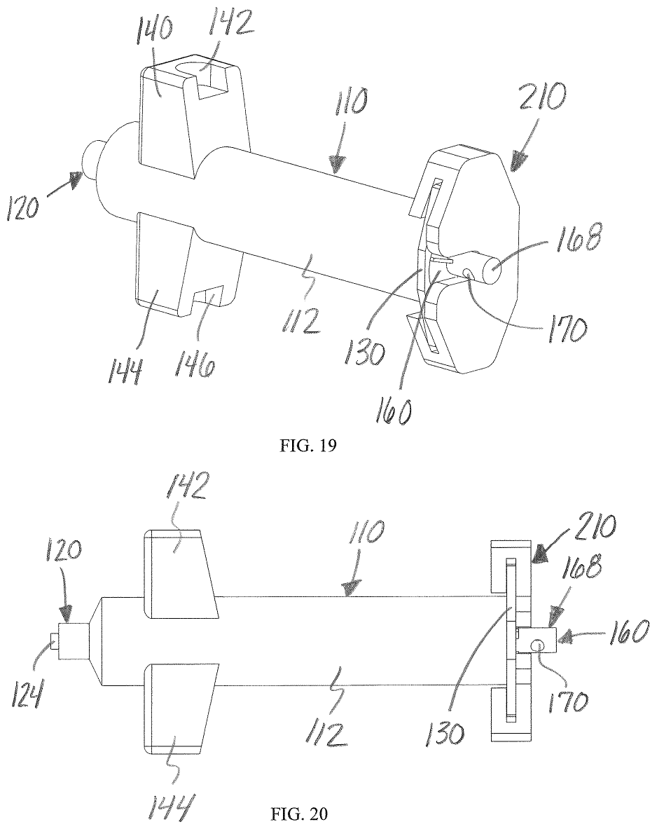

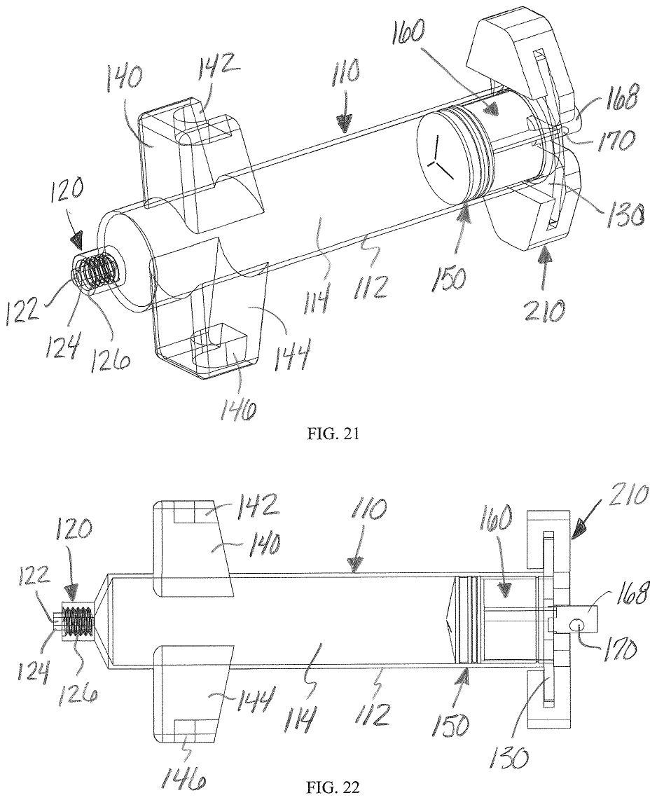

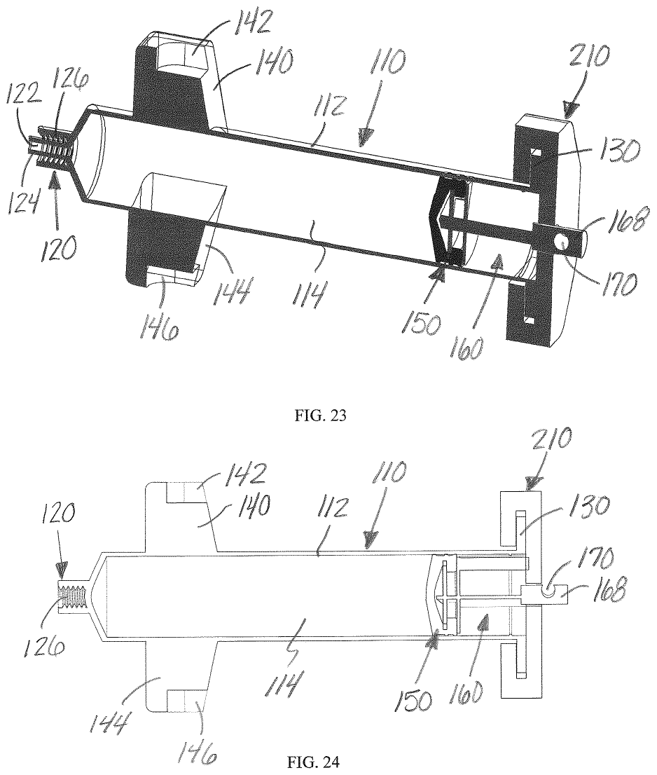

Referring to the drawings, wherein like reference numerals are used to indicate like or analogous components throughout the several views, and with particular reference to FIGS. 1-24, there are illustrated exemplary embodiments of syringes 100, 250, 300.

With reference to FIGS. 1-16 and 19-24, the syringe 100 is shown. The syringe 100 includes a housing 110, 230, a stopper 150, an inner plunger 160, an outer plunger 180, and a plunger pin 200. The stopper 150 is coupled to the inner plunger 160. The inner plunger 160 is coupled to the outer plunger 180 by a plunger pin 200. The coupled stopper 150, inner plunger 160, and outer plunger 180 is slidingly received within the housing 110, 230. The syringe 100 may also include a syringe cap or locking cap 210 for coupling to the housing 110, 230 during insertion into a centrifuge.

Referring now to FIGS. 1-14 and 19-24, the syringe 100 includes a first end 102 and a second end 104 opposite the first end 102. The syringe 100 includes a male housing 110 with a sidewall 112 and a chamber 114 extending into the sidewall 112 from a second end of the housing 110 toward a first end. The housing 110 includes a leur lock 120, for example, a male leur lock 120. The luer lock 120 extends from the first end of the housing 110. The luer lock 120 includes a tube 124 extending through the luer lock 120 surrounded by a coupling opening 126 positioned between the luer lock 120 and the tube 124. The tube 124 also includes a through hole 122 extending through the tube 124 into the chamber 114. The coupling opening 126 surrounds the tube 124 and extends into the luer lock 120 toward the first end of the housing 110. The coupling opening 126 may include, for example, threads for coupling to a female syringe housing, such as, housing 230.

The syringe 100 also includes a distal member 130 positioned at a second end of the housing 110. The distal member 130 includes a first wing 132 and a second wing 134. The first wing 132 extends out from a first side of the sidewall 112 of the housing 110 at the distal end. The second wing 134 extends out from a second side of the sidewall 112 of the housing 110 at the distal end. The first wing 132 extends out from a side opposite the second wing 134. The wings 132, 134 extend away from the sidewall 112 generally perpendicular. The syringe 100 further includes a first coupling arm 140 and a second coupling arm 142 extending away from the sidewall 112 of the housing 110 near the proximal or first end of the sidewall 112 of the housing 110. The first coupling arm 140 extends away from a first side of the sidewall 112 between the proximal end and the distal end of the sidewall 112 of the housing 110. The second coupling arm 144 extends away from a second side of the sidewall 112 between the proximal end and the distal end of the sidewall 112 of the housing 110. The first coupling arm 140 extends away from the sidewall 112 on a side opposite the second coupling arm 144. The first coupling arm 140 may also extend away from the sidewall 112 parallel to the first wing 132. The second coupling arm 144 may also extend away from the sidewall 112 parallel to the second wing 134. The first coupling arm 140 may include a first recess 142 inset into a portion of the first coupling arm 140. The first recess 142 may extend from a distal or second side of the arm 140 toward a proximal or first side of the arm 140. The first recess 142 may also extend from an exterior surface of the arm 140 towards the sidewall 112 of the housing 110. The first recess 142 may be, for example, configured or sized and shaped to be received and stabilized within a centrifuge machine. In addition, the second coupling arm 144 may include a second recess 146 inset into a portion of the second coupling arm 144. The second recess 146 may extend from a distal or second side of the arm 144 toward a proximal or first side of the arm 144. The second recess 146 may also extend from an exterior surface of the arm 144 towards the sidewall 112 of the housing 110. The second recess 146 may be, for example, configured or sized and shaped to be received and stabilized within a centrifuge machine.

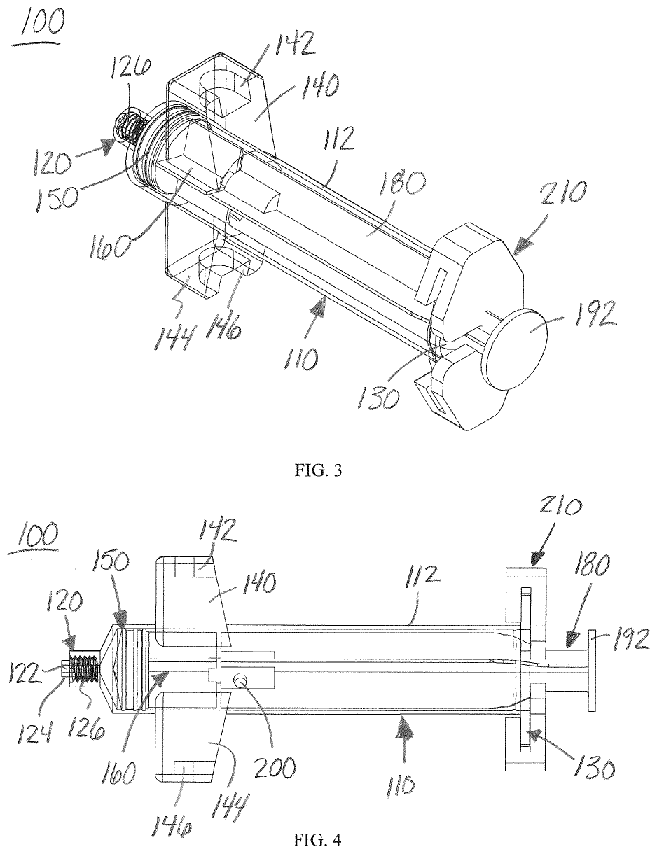

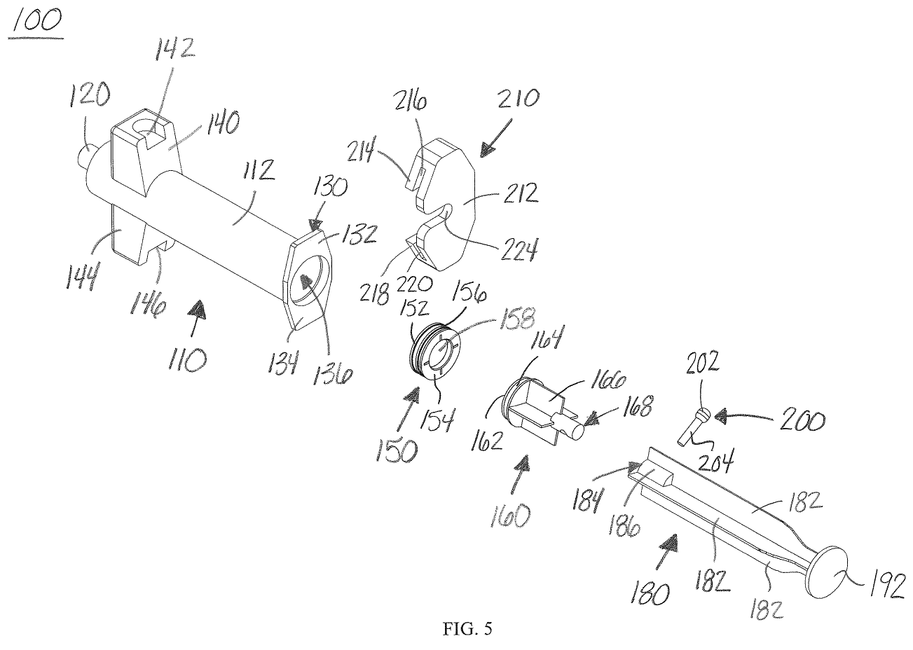

As best seen in FIGS. 3-12, the stopper 150 may include a first end 152 and a second end 154. The first end 152 is inserted into the housing 110. The stopper 150 also includes at least one flange member 156 extending circumferentially around the exterior surface of the stopper 150 between the first end 152 and the second end 154. The stopper 150 may further include an engagement opening 158 extending into the second end 154 of the stopper 150, as shown in FIG. 5. The engagement opening 158 may be, for example, configured or sized and shaped to receive at least a portion of the inner plunger 160. The stopper 150 may be, for example, made of an elastic material to allow for the inner plunger 160 to be inserted into the engagement opening 158.

The inner plunger 160 includes a proximal coupling end 162 for engaging the stopper 150. The inner plunger 160 further includes a groove 164 extending circumferentially around the inner plunger 160 near the proximal coupling end 162. The groove 164 may be, for example, configured or sized and shaped to receive a portion of the stopper 150 when the inner plunger 160 is coupled to the stopper 150. The inner plunger 160 also includes a plurality of projections 166 extending from the proximal coupling end 162 toward a second end of the inner plunger 160. The plurality of projections 166 may be, for example, equally spaced circumferentially around a bottom surface of the proximal coupling end 162 of the plunger 160. The plurality of projections 166 may be, for example, four projections as shown, although alternative numbers of projections are also contemplated to provide the desired amount of strength to the inner plunger 160 for removal and injection of blood related products. In addition, the inner plunger 160 includes a distal coupling rod 168 coupled to and extending from at least a portion of the plurality of projections 166. The rod 168 may be inset into at least a portion of the projections 166. The rod 168 extends to the end of the inner plunger 160. The rod 168 may include a through hole 170 extending through the rod 168 perpendicular to a longitudinal axis of the rod 168 extending between the proximal end and distal end of the plunger 160. The through hole 170 may be, for example, configured or sized and shaped to receive the plunger pin 200 to secure the inner plunger 160 to the outer plunger 180.

With continued reference to FIGS. 3-12, the outer plunger 180 also includes a plurality of projections 182 extending from a first end of the plunger 180 to the distal end member or injection member 192. The plurality of projections 182 may be, for example, equally spaced circumferentially around the member 192. The plurality of projections 182 may be, for example, four projections 182 as shown, although alternative numbers of projections are also contemplated to provide the desired amount of strength to the outer plunger 180 for removal and injection of blood related products. The projections 182 have different widths along the length of the projections 182. For example, the projections 182 may be wider at the first end of the plunger 180 than at the second end of the plunger 180. The change in width of the projections 182 may occur through a taper or gradual curvature at a point along the length of the plunger 180. In the depicted embodiment, the projections 182 decrease in width near the distal end of the plunger 180. In addition, the first end of the projections 182 may have the same total diameter as the diameter of the distal end member 192, while the second end of the projections 182 may have a total diameter that is smaller than the diameter of the distal end member 192.

In addition, the outer plunger 180 includes a proximal coupling end 184 with a coupling housing 186 extending from the first end of the outer plunger 180 toward the second end. The coupling housing 186 extends into the plunger 180 intermediate the plurality of projections 182. The coupling housing 186 may be, for example, in a middle of the projections 182 along a longitudinal axis of the plunger 180 extending between a first end and a second end of the plunger 180. The coupling housing 186 includes a cavity 188 extending into the housing 186 from a first end along the longitudinal axis of the plunger 180. The cavity 188 may be, for example, configured or sized and shaped to receive the distal coupling rod 168 of the inner plunger 160. The coupling housing 186 may further include an opening 190 extending into the housing 186 from an exterior surface and into the cavity 188. Thus, the opening 190 may extend into the coupling housing 186 perpendicular to the cavity 188. The opening 190 may be, for example, configured or sized and shaped to receive a portion of the plunger pin 200. The opening 190 may be, for example, positioned along the length of the coupling housing 186 to align with the position of the through hole 170 of the inner plunger 160. The opening 190 and through hole 170 are aligned for insertion of the plunger pin 200 through the inner and outer plungers 160, 180 to secure the inner plunger 160 to the outer plunger 180. The distal end member 192 may be, for example, a round or circular plate coupled to the distal end of the plurality of projections 182 to assist with translation of the plunger 160, 180 in the housing 110.

The plunger pin 200 may include a head 202 and a shaft 204. The shaft 204 may be coupled to and extend from the head 202. The head 202 may be, for example, spherical, round or the like to prevent the pin 200 from sliding completely through the through hole 170 and opening 190. In addition, the shaft 204 may be, for example, configured or sized and shaped to extend into the through hole 170 and opening 190 to secure the inner plunger 160 to the outer plunger 180, as shown in FIG. 4.

Finally, the syringe cap or locking cap 210 is shown in FIGS. 1-13. The cap 210 includes a base 212 with a first projection 214 extending from a first end of the base 212 and a second projection 218 extending from a second end of the base 212. The base 212 may have, for example, a polygonal shape, such as, an elongated octagon. The cap 210 may also include a first groove 216 extending between and separating a portion of the first projection 214 from the base 212. The first groove 216 may be, for example, configured or sized and shaped to receive a wing 132, 134 of the housing 110. The cap 210 may further include a second groove 220 extending between and separating a portion of the second projection 218 from the base 212. The second groove 220 may also be, for example, configured or sized and shaped to receive a wing 132, 134 of the housing 110. The first projection 214 may extend from a first end of the cap 210 toward the second end, while the second projection 218 may extend from a second end of the cap 210 toward the first end. The first projection 214 may be spaced apart from the second projection 218 by a slot 222 extending across the cap 210. The slot 222 may be, for example, configured or sized and shaped to receive the exterior surface of the housing 110. The cap 210 may further include a recess 224 inset into a portion of the base 212. The recess 224 may extend into the base from a first side toward the second side ending near a middle of the base 212.

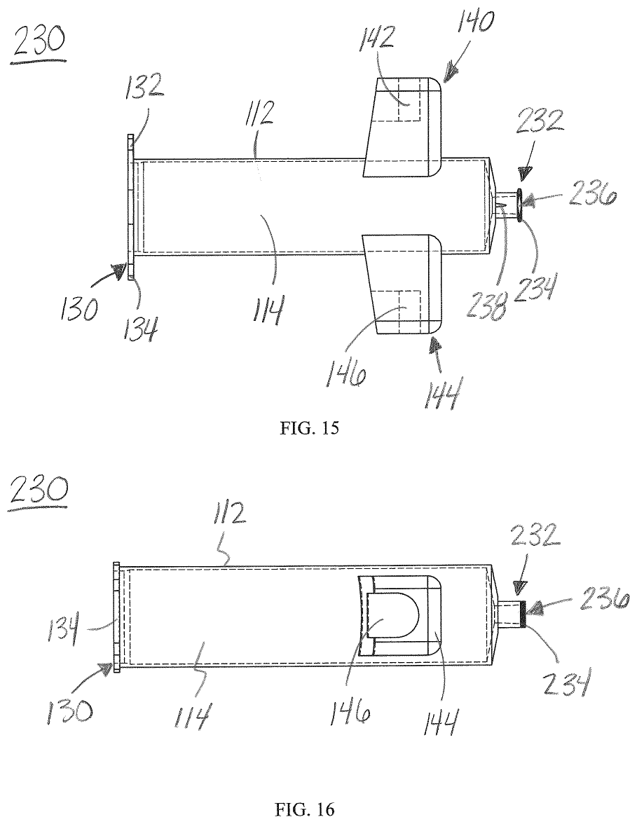

Referring now to FIGS. 15-16, an alternative housing 230 for syringe 100 is shown. The housing 230 is a female housing with a female luer lock 232 extending from the first or proximal end of the housing 230. The housing also includes a distal member 130 and coupling arms 140, 144 as described in greater detail above and which will not be described again here for brevity's sake. The female luer lock 232 includes a coupling end 234 for receiving a male luer lock, such as, male luer lock 120. The coupling end 234 may be, for example, inserted into the coupling opening 126 of the male luer lock 120. The female luer lock 232 may also include a through hole 236 extending into the coupling end 234 toward the chamber 114. A puncture mechanism 238 may be seated within the through hole 236 of the coupling end 234. The puncture mechanism 238 may be, for example, configured or sized and shaped to be inserted into the through hole 122 of the male luer lock 120. The male luer lock 120 may engage the female luer lock 230 to allow for transfer of blood products from one syringe 100 to another.

A method of using the syringe 100, 250 includes removing blood from the patient with a syringe 100, 250 including a male housing 110, 260. The outer plunger of the syringe 100, 250 may be removed. The syringe 100, 250 is then placed into the centrifuge to separate the red blood cells, buffy coat, and platelet-rich plasma. The syringe 100, 250 with the male housing 110, 260 is coupled to a syringe 100, 300 with a female housing 230, 310. The female syringe 100, 300 extracts the platelet-rich plasma from the male syringe 100, 250. The outer plunger of the female syringe 100, 300 is then removed before placing it into the centrifuge. Then, a second centrifuge is performed on the female syringe 100, 300 to separate the buffy coat and the platelet-rich plasma. The male syringe 100, 250 is then coupled to the female syringe 100, 300 and the buffy coat is removed from the female syringe 100, 300. Next, a syringe for administration to a patient is coupled to the female syringe 100, 300 and the platelet-rich plasma is moved to the syringe for injection into a patient.

Referring now to FIG. 17, another syringe system 250 is shown with a male luer lock 260. Referring now to FIG. 18, another syringe system 300 is shown with a female luer lock 310. Both syringe systems 250, 300 include coupling arms 270, 320, respectively, for engaging a centrifuge machine.
Clause 1. A syringe, comprising: a housing, comprising: a first coupling arm extending from a first side of the housing; and a second coupling arm extending from a second side of the housing, wherein the first side is opposite the second side; and at least one plunger slidingly received within the housing.
Clause 2. The syringe of clause 1, wherein the housing further comprises: a male luer lock extending from the first end of the housing.
Clause 3. The syringe of any one of clauses 1-2, wherein the housing further comprises: a female luer lock extending from the first end of the housing.
Clause 4. The syringe of any one of clauses 1-3, wherein the first coupling arm comprises: a first recess inset into the first coupling arm from a second end.
Clause 5. The syringe of any one of clauses 1-4, wherein the second coupling arm comprises: a second recess inset into the second coupling arm from a second end.
Clause 6. The syringe of any one of clauses 1-5, wherein the at least one plunger comprises: an inner plunger; and an outer plunger coupled to a second end of the inner plunger; wherein the coupled inner plunger and outer plunger are slidingly received within the housing.
Clause 7. The syringe of any one of clauses 1-6, wherein the inner plunger is secured to the outer plunger by a pin.
Clause 8. The syringe of any one of clauses 1-7, further comprising: a stopper coupled to a first end of the inner plunger.
Clause 9. The syringe of any one of clauses 1-8, wherein the housing further comprises: a distal member extending from a second end of the housing.
Clause 10. The syringe of any one of clauses 1-9, the distal member comprising: a first wing extending from the first side of the housing; and a second wing extending from the second side of the housing.
Clause 11. The syringe of any one of clauses 1-10, further comprising: a syringe cap removable coupled to the distal member.
Clause 12. The syringe of any one of clauses 1-11, wherein the syringe cap comprises: a base; a first projection coupled to and extending from the base; a first groove extending between the base and at least a portion of the first projection; a second projection coupled to and extending from the base; and a second groove extending between the base and at least a portion of the second projection.
Clause 13. The orthotic device of any one of clauses 1-12, wherein the first wing engages the first groove and the second wing engages the second groove when the syringe cap is coupled to the housing.
Clause 14. A syringe system, comprising: at least one first syringe, comprising: a housing, comprising: a first coupling arm extending from a first side of the housing; and a second coupling arm extending from a second side of the housing, wherein the first side is opposite the second side; and at least one plunger slidingly received within the housing; at least one second syringe; and at least one syringe cap for removable coupling to at least one of the at least one first syringe and the at least one second syringe.
Clause 15. The syringe system of clause 14, wherein the at least one first syringe is a male syringe and the at least one second syringe is a female syringe.
Clause 16. The syringe system of any one of clauses 14-15 device may include a housing, comprising: a first coupling arm extending from a first side of the housing; and a second coupling arm extending from a second side of the housing, wherein the first side is opposite the second side. A device may include at least one plunger slidingly received within the housing.5, wherein the at least one second syringe comprises: a housing, comprising: a first coupling arm extending from a first side of the housing; and a second coupling arm extending from a second side of the housing, wherein the first side is opposite the second side; and at least one plunger slidingly received within the housing.

As may be recognized by those of ordinary skill in the art based on the teachings herein, numerous changes and modifications may be made to the above-described and other embodiments of the present disclosure without departing from the scope of the disclosure. The components of the device as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the devices may include more or fewer components or features than the embodiments as described and illustrated herein. Accordingly, this detailed description of the currently-preferred embodiments is to be taken in an illustrative, as opposed to limiting of the disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiment. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. A syringe, comprising:
a housing, comprising:
a first coupling arm extending from a first side of the housing; and
a second coupling arm extending from a second side of the housing, wherein the first side is opposite the second side; and
at least one plunger slidingly received within the housing.

2. The syringe of claim 1, wherein the housing further comprises:
a male luer lock extending from the first end of the housing.

3. The syringe of claim 1, wherein the housing further comprises:
a female luer lock extending from the first end of the housing.

4. The syringe of claim 1, wherein the first coupling arm comprises:
a first recess inset into the first coupling arm from a second end.

5. The syringe of claim 4, wherein the second coupling arm comprises:
a second recess inset into the second coupling arm from a second end.

6. The syringe of claim 1, wherein the at least one plunger comprises:
an inner plunger; and
an outer plunger coupled to a second end of the inner plunger;
wherein the coupled inner plunger and outer plunger are slidingly received within the housing.

7. The syringe of claim 6, wherein the inner plunger is secured to the outer plunger by a pin.

8. The syringe of claim 6, further comprising:
a stopper coupled to a first end of the inner plunger.

9. The syringe of claim 1, wherein the housing further comprises:
a distal member extending from a second end of the housing.

10. The syringe of claim 9, the distal member comprising:
a first wing extending from the first side of the housing; and
a second wing extending from the second side of the housing.

11. The syringe of claim 9, further comprising:
a syringe cap removable coupled to the distal member.

12. The syringe of claim 11, wherein the syringe cap comprises:
a base;
a first projection coupled to and extending from the base;
a first groove extending between the base and at least a portion of the first projection;
a second projection coupled to and extending from the base; and
a second groove extending between the base and at least a portion of the second projection.

13. The orthotic device of claim 12, wherein the first wing engages the first groove and the second wing engages the second groove when the syringe cap is coupled to the housing.

14. A syringe system, comprising:
at least one first syringe, comprising:
a housing, comprising:
a first coupling arm extending from a first side of the housing; and
a second coupling arm extending from a second side of the housing,
wherein the first side is opposite the second side; and
at least one plunger slidingly received within the housing;
at least one second syringe; and
at least one syringe cap for removable coupling to at least one of the at least one first syringe and the at least one second syringe.

15. The syringe system of claim 14, wherein the at least one first syringe is a male syringe and the at least one second syringe is a female syringe.

16. The syringe system of claim 15, wherein the at least one second syringe comprises:
a housing, comprising:
a first coupling arm extending from a first side of the housing; and
a second coupling arm extending from a second side of the housing, wherein the first side is opposite the second side; and
at least one plunger slidingly received within the housing.

17. A method of using a syringe system, comprising:
obtaining a syringe system;
removing blood from a patient with a first syringe of the syringe system, wherein the first syringe includes two coupling arms positioned on an exterior surface of the first syringe opposite each other;
placing the syringe into a centrifuge machine; and
coupling the first syringe with a second syringe and extracting platelet-rich plasma from the first syringe.

18. The method of claim 17, wherein the first syringe has a male luer lock and the second syringe has a female luer lock.

19. The method of claim 17, wherein the second syringe includes two coupling arms positioned on an exterior surface of the second syringe opposite each other.

20. The method of claim 19, further comprising:
inserting the second syringe into the centrifuge machine;
coupling the second syringe to a third syringe and extracting platelet-rich plasma from the second syringe.
